(19) United States
(12) Patent Application Publication
(10) Pub. No.: US 2005/0021372 A1
(43) Pub. Date: Jan. 27, 2005

(54) Interactive motivation systems and methods for self-care compliance

(75) Inventors: Tarjei Sigurd Mikkelsen, Cambridge, MA (US); Vikram Sheel Kumar, Boston, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Dimagi, Inc., Boston, MA (US)

(21) Appl. No.: 10/874,482

(22) Filed: Jun. 23, 2004

Related U.S. Application Data
(60) Provisional application No. 60/490,379, filed on Jul. 25, 2003.

(51) Int. Cl. G06F 17/60; A61B 5/00

(52) U.S. Cl. 705/2; 600/300

(57) ABSTRACT

Disclosed are methods and systems that include receiving data associated with the compliance of a user with a self-care regimen, evaluating the data to determine a level of compliance of the user with the self-care regimen, and using at least one executable processor application to provide the user with an incentive based on the determined level of compliance.

### CLAIM OF PRIORITY

This application claims priority to U.S. Ser. No. 60/490,379, filed on Jul. 25, 2003, the contents of which are incorporated herein by reference in their entirety.

### BACKGROUND

(1) Field

The disclosed methods and systems relate to health education and personal health care, and more particularly to systems and methods of evaluating and rewarding compliance with a self-care regimen.

(2) Description of Relevant Art

Whereas major technological advances have led to improvements in an ability to treat and heal acute medical conditions, there are a number of chronic diseases that have no current cure; however, a person suffering from these conditions can often live a productive and healthy life if the condition is identified and managed.

Compliance with medical self-care regimens can have a strong positive effect on the health and well-being of patients with a variety of chronic disease conditions. For example, the development of diabetes complications is directly related to the level of glycemic control in persons with diabetes. It has been shown that the frequency of blood glucose monitoring can significantly impact the level of glycemic control. More frequent blood glucose monitoring yields better glycemic control with lower average blood glucose levels, which translates to a fewer long term complications.

Noncompliance with self-treatment regimens depending on inhalation devices is a common cause of hospitalization for asthma patients. Asthma can be effectively managed using inhalation devices, but if noncompliant behavior remains unrecognized, these individuals are at risk for recurrent emergency care and death. While new combination medications make taking medications easier, the problem of low motivation for a treatment remains formidable.

Despite the objective benefits of following strict self-care regimens, there are numerous negative personal and social factors that can weigh heavily on chronic disease patients, leading to noncompliance. These factors include the monotony of following daily treatment routines, the desire to live without restrictions on diet and activities, and social stigmas attached to disease and disabilities.

Traditional methods to help chronic disease patients remain motivated and compliant with their self-monitoring and self-treatment regimens focus on delivering health education in various forms and providing simpler monitoring technologies. While these methods and systems may inform the patient of the risks of noncompliance and lessen the burden of self-care regimens, they are often not sufficient to keep patients motivated over extended periods of time in the face of said negative factors.

In addition, portable computers have become important entertainment and lifestyle tools for a wide variety of people in various forms, such as personal digital assistants, cellular phones and portable video game units. They therefore provide opportunities for integrating motivational and educational systems into the daily life of a chronic disease patient, with minimal need for introducing additional technologies or routines.

### SUMMARY

The disclosed methods and systems include a motivational method comprising interactive software programs and games that evaluate usage data from health sensors and reward users that comply with a prescribed self-care regimen with higher scores and/or access to restricted features. Users that do not comply may receive lower scores, which may cause a virtual character to be deleted, and/or to be presented in a condition that may be based on the user's actual condition should the user continue non-compliance with the self-care regimen. In some embodiments, the data received by the methods and systems can be provided via wired and/or wireless networks using processor controlled devices to communicate information between such devices to eliminate false entries and/or inaccurate data that may occur with manual entry. In other embodiments, manual entry, and/or combinations of automatic/electronic data reception and manual entry, can be used.

The disclosed methods and systems integrate incentives that encourage compliance into entertainment and lifestyle programs that are not exclusively focused on the user's medical condition. The disclosed methods and systems can thus provide motivational support and evaluate compliance levels based on collected objective measures and/or data, such as health sensor usage data, rather than, for example, relying on potentially inaccurate self-reporting.

The disclosed methods and systems thus provide a method that motivates compliance with medical self-care regimens, a method of health education that is suited to the formation of healthy habits, and/or a method for maintaining accurate records of health sensor data for use in chronic disease management.

Disclosed are systems and methods that include receiving data associated with the compliance of a user with a self-care regimen, evaluating the data to determine a level of compliance of the user with the self-care regimen, and using at least one executable processor application to provide the user with an incentive based on the determined level of compliance. The incentive can include changing a condition of a virtual entity/character, where the changed condition is associated with the determined level of compliance. The changed condition can be associated with the user's condition based on the determined level of compliance and the self-care regimen.

In some embodiments, the incentive can include a reward to the user, where the reward can include access to an additional processor application(s), an enhanced feature(s) of a processor application(s), and/or an improved score. The change in a user score can affect at least one of an activity, physical condition, and characteristic (e.g., a personality trait, a physical characteristic, an ability within the application(s), etc.) of a virtual character associated with the processor application(s). In one embodiment, the score can affect, positively or adversely, a health and/or a happiness of a virtual pet.

Receiving data can include receiving data from a blood glucose level measuring device, an inhalation device, an electronic weighing scale, a blood pressure measuring device, a heart rate monitoring device, an accelerometer, a blood clotting measurement device, a voice recording device, and/or a medication dispensing device. Accordingly, evaluating the data can include determining compliance with a diabetes management self-care regimen, a breathing disorder self-care regimen, a weight management self-care regimen, a cardiovascular condition self-care regimen, a hypertension self-care regimen, a physical activity self-care regimen, a bleeding disorder self-care regimen, a mood disorder self-care regimen, and/or a self-medication self-care regimen. Evaluating the data can also include establishing at least rule describing respective conditions to be satisfied in attaining respective levels of compliance with the self-care regimen, and evaluating the rules based on the data. In some embodiments, the rules can be logical rules.

In some embodiments, the self-care regimen is a medical regimen. Further, the executable processor application(s) can reside on a personal digital assistant, a cellular telephone, a wireless personal communications device, a portable video game device, and/or a digital watch. The received data can thus be received via a wired and/or wireless communications network, via, for example, digital devices.

In one embodiment, the methods and systems include a computer game tangibly stored on a computer-readable medium and operable to cause a processor to receive data relevant to the compliance of a user with a self-care regimen, evaluate the data to determine a level of compliance with the self-care regimen, update at least one of an activity, a physical condition, and a characteristic of at least one interactive virtual entity, based on the determined level of compliance, to obtain a condition of the virtual entity, and, display the virtual entity to the user to convey the condition of the virtual entity to the user. The computer game can also include instructions to provide a choice of actions to the user, the actions for modifying the condition of the virtual entity. The instructions can include instructions to mark the virtual entity for deletion, and/or otherwise delete the virtual entity/character, based on a comparison of the condition to a threshold.

The instructions to update can include instructions to determine a time interval since the condition was obtained, and update the condition based on the time interval. The instructions can also include instructions to convey the condition of the virtual entity consistent with the self-care regimen effects on the user.

In one embodiment, the methods and systems include a system for improving a person's compliance with a self-care regimen that can include an input mechanism and/or receiver (e.g., network port, antenna, keyboard, joystick, user interface) for receiving data relevant to the compliance with the self-care regimen from one or more devices, and processor instructions for evaluating the data received by the input device to determine the compliance with the self-care regimen, allowing the person to interact with one or more computer applications, and for providing feedback to the person relative to the compliance with the self-care regimen, where the feedback conditions, alters the operation, and/or affects the processor executable application(s) based on the determined level of compliance.

The system can include one or more storage mechanisms for storing the data for future retrieval. Accordingly, the self-care regimen can include a medical regimen, and the received data can be received from one or more health monitoring devices. The input device can thus include a data communications interface to and/or receiver for one or more health monitoring devices that may include a blood glucose level measuring device for diabetes management, an inhalation device for management of breathing disorders, an electronic weighing scale for weight management, a blood pressure measuring device for management of cardiovascular conditions and hypertension, a heart rate monitoring device for management of cardiovascular conditions, an accelerometer for monitoring a physical activity, a blood clotting measurement device for management of bleeding disorders, a voice recording device for management of mood disorders, and a medication dispensing device for management of self-medication, for example.

As a reward for compliance with the self-care regimen, the feedback can condition the processor application(s) to provide access to one or more additional computer applications, to provide enhanced features of for the computer/processor application(s) being executed by the user, and/or to provide a change in score in a computer application being executed by the person/user. For example, improved scores can include improved health and/or improved happiness of a virtual pet/character(s), while lower scores can include decreased health and/or decreased happiness of a virtual pet/character(s), where such health and happiness may reflect the person/user's compliance with the self-care regimen in that the virtual entity's condition can be reflective of the user/person's compliance with the self-care regimen.

The storage can reside on a read-only memory, a random-access memory chip, a computer hard-drive, an optical disc, and/or a compact disc, for example. The system can include a network interface, wherein at least a portion of the storage resides on a server connected to the network interface via a communications network. The storage can include a database and/or computer instructions. The interaction can include a personal digital assistant, a wireless personal communication device, a portable video game device, and/or a processor in a digital watch.

Also disclosed is a computer readable medium including processor-readable instructions for implementing the methods and systems described herein.

These and other objects and advantages will become more apparent after consideration of the ensuing description and the accompanying drawings.

### BRIEF DESCRIPTION OF THE DRAWINGS

FIG. 1 shows schematically some mediating factors that can be used to encourage compliance and/or promote healthy behavior in one method of the methods and systems disclosed herein;

FIG. 2 shows a flowchart of a relationship between the patient's goals and influencing factors in one system according to the disclosed methods and systems;

FIG. 3 shows a flowchart for one embodiment of the disclosed methods and systems;

FIG. 4 shows an example of a logical rule for evaluating compliance with a diabetes self-monitoring regimen used by an instance of according to an embodiment of FIG. 3;

FIG. 5 shows a flowchart for an embodiment of the disclosed methods and systems;

FIG. 6 shows an example of a logical rule for evaluating compliance with a weight-management self-monitoring regimen used by an instance of the embodiment of FIG. 5;

FIG. 7 shows an embodiment of the system in a local evaluation approach;

FIG. 8 shows an embodiment of the system in a server evaluation approach; and,

FIG. 9 shows a flowchart of the relationship between the patient's goals and influencing factors in a motivation system.

### DESCRIPTION

To provide an overall understanding, certain illustrative embodiments will now be described; however, it will be understood by one of ordinary skill in the art that the systems and methods described herein can be adapted and modified to provide systems and methods for other suitable applications and that other additions and modifications can be made without departing from the scope of the systems and methods described herein.

Unless otherwise specified, the illustrated embodiments can be understood as providing exemplary features of varying detail of certain embodiments, and therefore, unless otherwise specified, features, components, modules, and/or aspects of the illustrations can be otherwise combined, separated, interchanged, and/or rearranged without departing from the disclosed systems or methods. Additionally, the shapes and sizes of components are also exemplary and unless otherwise specified, can be altered without affecting the scope of the disclosed and exemplary systems or methods of the present disclosure.

The present disclosure is directed to interactive, positive, motivation systems and methods that can encourage users to comply with a predetermined medical self-care regimen, for example, as prescribed by a physician or counselor. The systems and methods can include one or more interactive programs or games executing on a processor device (e.g., portable computer, PDA, processor associated with and/or incorporated into a health sensor, etc.) and communicating the time and value of health-related sensor readings to the computer. A user who operates the health sensors in compliance with the specified self-care regimen can be rewarded with an improved score or access to new features in the program and/or game. The scores and/or other data associated with the interactive program, game, etc., can further be configured to be communicated to a parent, physician, peer/friend (e.g., multi-user game) and/or other.

The present description discloses the methods and design strategies incorporated into a video game and a generic motivational tool; however, the methods and systems described herein can be incorporated in other forms of games, including electronic games, computer games, and/or other interactive games, and may further be incorporated in motivational tools including incentive programs, positive reinforcement and/or other tools for motivating compliance of a user to a regimen (e.g., non-health-related self-care regimens).

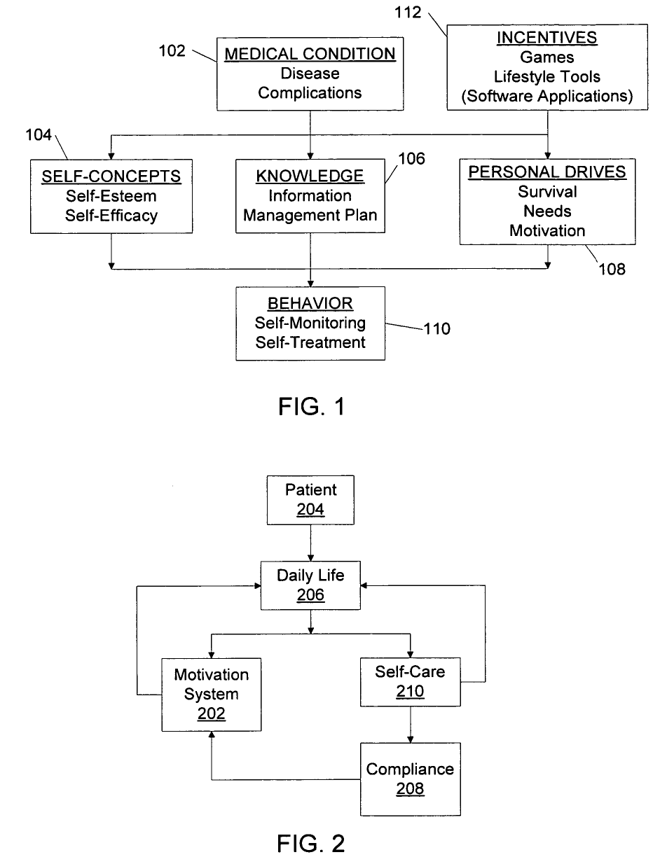

FIG. 1 shows schematically some mediating factors that can be used to encourage compliance and promote healthy behavior by the present disclosed methods and systems. A medical condition 102, such as the chronic diseases diabetes and asthma, can impact the self-concepts 104 of a patient, often negatively; can create a need for information and management plans to treat the condition 106; and can activate personal drives 108, such as the desire to live healthy. The interactions of these three mediating factors can impact the level of compliance to a self-care regimen displayed by the patient. For example, negative self-esteem (self-concept 104) and boredom (personal drive 108) may effectively cancel the effects of strong health education (information 106), leading to noncompliance (behavior 110). The present disclosed methods and systems can provide incentives 112 through games and lifestyle tools, e.g., software applications, that can positively influence the patient's motivation to be compliant (personal drive 108), e.g., by providing incentives, for example, that meet the patient's need for entertainment (personal drive 108), thereby increasing the likelihood of compliance (behavior 110).

Computer systems that incorporate incentives for compliance into useful or entertaining applications, or that restrict access to such applications in the case of noncompliance, have great potential for motivating patients beyond the scope of didactic health education. Systems relying on self-reporting can be ineffective and can provide opportunities for cheating. Quantifying a user's level of compliance through objective means can serve to alleviate such problems. Objectively quantifying a user's level of compliance can be achieved by coupling the system with one or more health sensors, such as a blood glucose measuring device, that automatically record and store readings and usage data. Such devices are currently available to patients who wish to automatically record their health data for use in improving self-care regimens and monitoring the progression of their medical conditions. The methods and systems disclosed herein can extend the usefulness of these sensors by integrating their record-keeping capabilities with a system that a patient, who otherwise may not be motivated or meticulous enough to track and store their sensor readings, can find attractive to use.

Various means, such as persistent incentives, can be used to maintain patients sufficiently motivated to follow strict, daily, self-care regimens. FIG. 2 shows a flowchart of the interactions of a health motivation application, such as 112 of FIG. 1, that can be integrated into the patient's daily life and routine. The motivation system 202 disclosed herein can be carried with a patient 204 throughout the patient's daily routine 206, and can respond directly to the patient's compliance 208 with the patient's self-care regimen 210. For comparison, FIG. 9 shows a flowchart of the interactions of a motivation application 902, such as an independent educational session, that interacts with the daily life 904 of the patient 906 independently from the patient's self-care regimen 908.

For convenience and explanatory purpose, the systems and methods can be described herein with reference to exemplary systems and methods for use in motivating compliance with a self-care regimen for a diabetic patient and a weight monitoring regimen. For the illustrated diabetic self-care regimen, the systems and methods can include a game featuring a virtual pet. For the illustrated weight monitoring regimen, the systems and methods can include providing access to games and/or programs having incentive value to the patient. However, the systems and methods described herein are not to be limited to the embodiments disclosed herein, and can be applicable to motivating compliance with other periodic regimens, or routines, and can include other applications and/or means for providing rewards and/or incentives for compliance with the regimen, and that additions, modifications, and/or other changes to the receiver, input, processing, and/or output to accommodate such other regimens, applications and/or means are contemplated by the systems and methods described herein and can be made by those skilled in the art.

An exemplary virtual pet game can be configured for operation on a personal digital assistant (PDA) and can be directed to patients with insulin-dependent diabetes. Modifications and/or changes to accommodate other platforms, including cellular telephones, laptops, personal computers, etc., to accommodate patients with other conditions for which monitoring is desirable, and to accommodate patients of different age ranges, are contemplated. In some embodiments, the disclosed methods and systems can be integrated with a sensor/measurement device such that a single device can provide measurements and interactive aspects as provided herein (e.g., glucose meter equipped with a display and/or processor instructions for effecting the disclosed methods and systems.) Generally, the compliance level of the user, or patient, can directly influence the game-play.

For the virtual pet diabetes game, the compliance level can be evaluated from usage data from a blood glucose measuring device. The game can feature a virtual pet that lives on a portable computer screen. The condition of the pet can be described by a set of interrelated variables, including health, mood, hunger, sleepiness, energy and desire for attention. The pet can abandon the patient, or player, if the pet becomes unhealthy or feels unhappy. Noncompliance with a pre-programmed blood glucose monitoring plan can have adverse effects of the pet's mood and health. To maintain the health and happiness of the pet, the player can give the virtual pet frequent and appropriate attention, and can demonstrate compliance with the blood glucose monitoring plan.

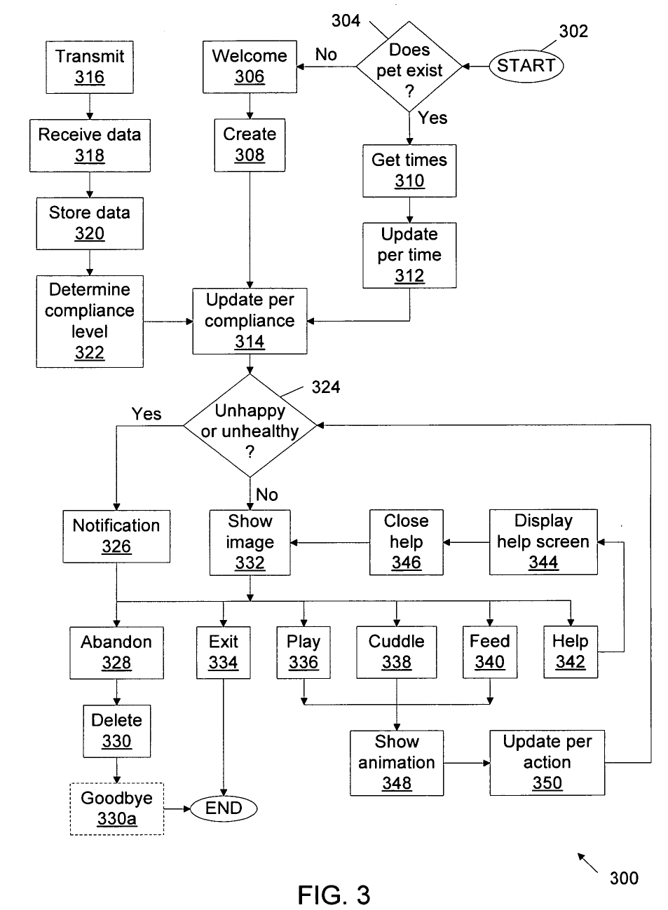

FIG. 3 shows a simplified flowchart for an embodiment of a virtual pet game, application, or method of motivating compliance 300, as described herein. The illustrated game 300 can be designed so as to be capable of intermittent play. When the player starts 302 the game application 300, the application 300 can check 304 whether the session is a continuation of a previous session, or whether a new pet can be created. When the player starts the game application 300 for the first time, or no current pet exists in the game database, the player can be presented with a welcome screen 306 that can instruct the player how to play the game. The player can create 308 a virtual pet by selecting a pet species (e.g., puppy, kitten, goldfish, parakeet, or fictional entity), naming it, and selecting a personality (energetic, shy, friendly or lazy). The personality of the pet can determine how often the player interacts with it, and what kinds of interaction can make the virtual pet happy or healthy. The pet's condition variables, including health, mood, hunger, sleepiness, energy, activity, desire for attention, and/or other characteristic, can be set to default values.

If the game 300 is started with an existing pet in its database, the game 300 can determine 310 the current time, and how long it has been since the player's previous game session. The pet's condition variables can be adjusted in proportion to these values, in a manner consistent with its personality, so as to update the pet's mood and/or health 312. For example, if it is daytime and an energetic pet has not been interacted with for over an hour, the game can increase the pet's energy level, and decrease its mood level. Similarly, pets can have increased sleepiness levels at night, and increased hunger levels if they have not been fed for over 3 hours during day-time.

After a new pet has been created at 308, or the condition of an existing pet has been updated at 312, the game 300 can proceed to a compliance-influenced determination or update of the pet's mood and/or health 314. For the exemplary diabetic patient, a daily diabetes self-monitoring regimen can include the use of a digital glucometer, such as are available from a number of medical device companies, to track blood glucose levels. The time and value of the glucometer readings can be input, received by, and/or provided 316, and/or transmitted to the PDA or other processor, on which the game 300 executes, e.g., via a wireless communication interface. As the data from the glucometer is received 318, it can be entered 320 into a database. Application 300 can use the contents of this database to determine 322 whether the patient has been compliant with the prescribed self-monitoring regimen and determine a level of compliance for the patient.

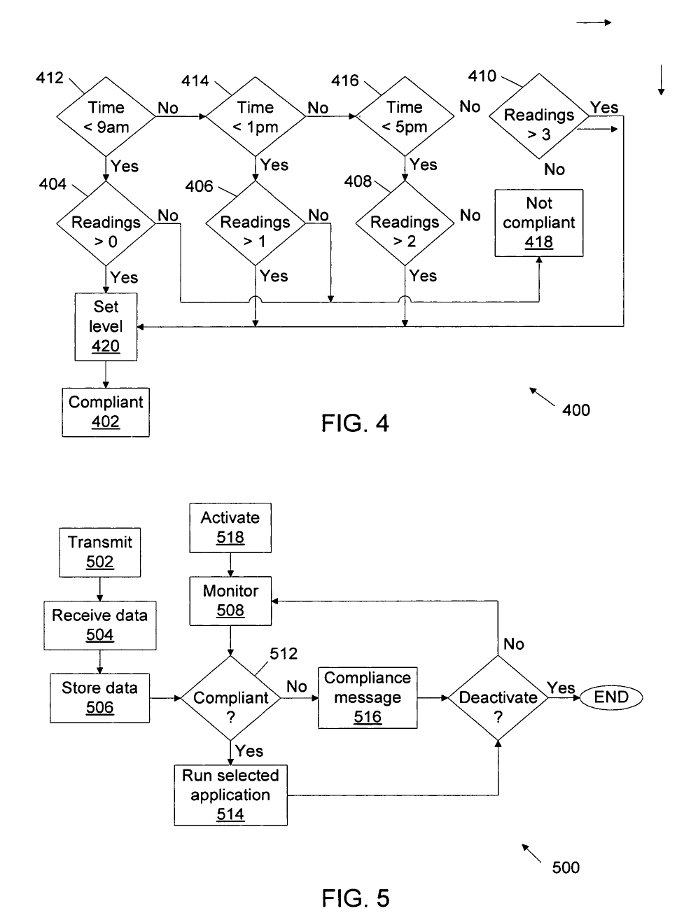

FIG. 4 shows a flowchart for an exemplary rule 400 that can be used to determine if the patient is compliant with his self-monitoring regimen at a particular time of the day. The illustrated rule 400 considers the player and/or patient compliant 402 if a minimum number of blood glucose readings (404, 406, 408, 410), as determined by the time of day (412, 414, 416), have been entered into the database. If not, the patient is considered non-compliant 418. In addition, compliance levels can be set 420 based on the compliance determinations for the various number of glucose readings (404, 406, 408, 410). Rules, such as rule 400, can be developed by qualified medical experts for an individual patient, or a group of similar patients, can be more or less detailed, and/or can depend on additional and/or different variables, e.g., the values of the readings of the glucometer, than shown for exemplary rule 400.

Referring back to FIG. 3, compliance-influenced update 314 can use the compliance level determined at 322 to update the pet's mood and/or health. For example, if the patient is determined to be compliant, the mood level of the pet can be improved as a positive reward. However, if the player is not compliant, the mood and health levels of the pet can be decreased. The amount of improvement or decline can depend on determined compliance levels. Because one object of the illustrated game 300 is to maintain the virtual pet happy and healthy, compliance-influenced update 314 of the game 300 can establish a direct link between beneficial self-monitoring behavior and the patient's entertainment goals.

The FIG. 3 game 300 can examine the mood and health levels of the virtual pet 324. If the pet mood and health levels are below predetermined threshold levels, for example i.e., the pet is both unhappy and unhealthy, the game 300 can provide 326 a notice to the patient and can proceed to abandon 328 the pet. Alternatively, the notice can provide instructions to the patient on the processes and/or actions the patient can take to stop the abandonment and the game 300 can await further action by the patient. If abandonment occurs, the game 300 can delete 330 the pet and optionally can display 330a a goodbye message from the pet. The game 300 can then end. To play again, in the illustrated embodiment, the patient must restart the game and create a new pet (304, 306, 308). If the pet's mood and/or health are above the predetermined thresholds 324, the game 300 can show an image or animation of the virtual pet 332.

The image and/or animation can include facial expressions and body language of the pet that can represent the pet's current mood and health level. For example, a happy and energetic pet can smile and/or look anticipant, whereas an unhappy pet can growl and/or look away from the player. Additional numeric indicators on the screen can display the current values of the pet's condition variables. The image can include a menu from which the player can choose, e.g., by pressing one of several labeled buttons on a main game screen, one or more actions to take relative to the pet and/or the game 300. In the exemplary game 300, the actions can include abandoning 328 the pet, as described above, exiting 334, playing 336 with the pet, cuddling 338 the pet, feeding 340 the pet, and seeking help 342 for the game 300. Depending on the chosen pet and personality, the menu can include other actions related to the pet's health and/or mood. For example, actions for a pet fish can include changing the water in the fish tank, while actions for a pet dog can include taking the dog for a walk. If the player chooses to seek help 342, the game 300 can display 344 a screen containing instructions. The player can return to the image or animation 332 upon closing 346 the help screen.

If the player chooses one of the action buttons related to the pet's health and/or mood, e.g., playing 336, cuddling 338, or feeding 340, an animation of the selected action can be shown 348 on the game screen. Different actions can involve different levels of participation by the patient and provide varying levels of improvement in the pet's health and/or mood. For example, feeding 340 can involve a obtaining food, cleaning a bowl, mixing ingredients, etc., while cuddling 338 can include stroking the pet a number of times. The virtual pet's condition variables can be updated 350 according to whether the action taken meets the pet's current needs. For example, if the patient selects cuddling 338 when the pet had a high desire for attention, game 300 can decrease the pet's desire for attention and increase the pet's happiness. On the other hand, if the player selects playing 336 when the pet is very sleepy, the pet's health and/or mood can be decreased. After the pet's condition variables have been adjusted, game 300 can return to 324 for determining the pet's mood and health levels.

The illustrated game 300 can thus leverage a patient's interest and emotional attachment to the virtual pet and/or other object/character to encourage healthy behavior. It will be obvious to one skilled in the art that game 300 can be implemented in a number of other and more complex variations, including substituting the pet with, for example, a virtual person or family, character, and/or object, and/or including different and/or expanded sets of relevant variables and interrelations. Further, game 300 can include an educational component that can be added by incorporating condition variables and interactions that model, emulate, simulate, and/or otherwise reflect the medical condition of the player or patient.

An embodiment of the present systems and methods can include applications providing access to games and/or programs having incentive value to the patient. Such applications can be configured for operation on a personal digital assistant (PDA) and/or cellular telephone, for example, and can be directed to users, or patients requiring and/or desiring a monitoring regimen. Modifications and/or changes to accommodate other platforms, including laptops, personal computers, etc., are contemplated. For convenience and explanatory purpose, an application related to a weight monitoring regimen can be described, though such applications can be configured to provide motivation for compliance with other forms of regimens and/or self-care programs.

FIG. 5 illustrates a flowchart for an application 500 for an exemplary weight monitoring regimen, wherein the application 500 can grant or deny access to a selected set of applications, based on the user's current compliance level. Time and weight measurements, e.g., from an electronic weighing scale, can be input or transmitted 502 to the PDA, or other processor on which the application(s) 500 executes, e.g., via a wireless communication interface. As the data from the weighing scale is received 504, it can be entered 506 into a database. Application 500 monitors 508 user activity on the PDA or processor. When the user or patient attempts to start one of the applications from the selected set of applications, as at 510, application 500 can evaluate 512 the patient's compliance level from the data stored in the database. If the patient is judged to be compliant, the requested application can be started 514. Otherwise, a message can be provided 516 to the patient instead of starting the requested application. The message, or other form of communication with the patient, can explain that the patient is noncompliant and can provide instructions on achieving compliance. After starting the requested application(s) 514, or providing the message 516, the application(s) 500 can return to monitoring 508.

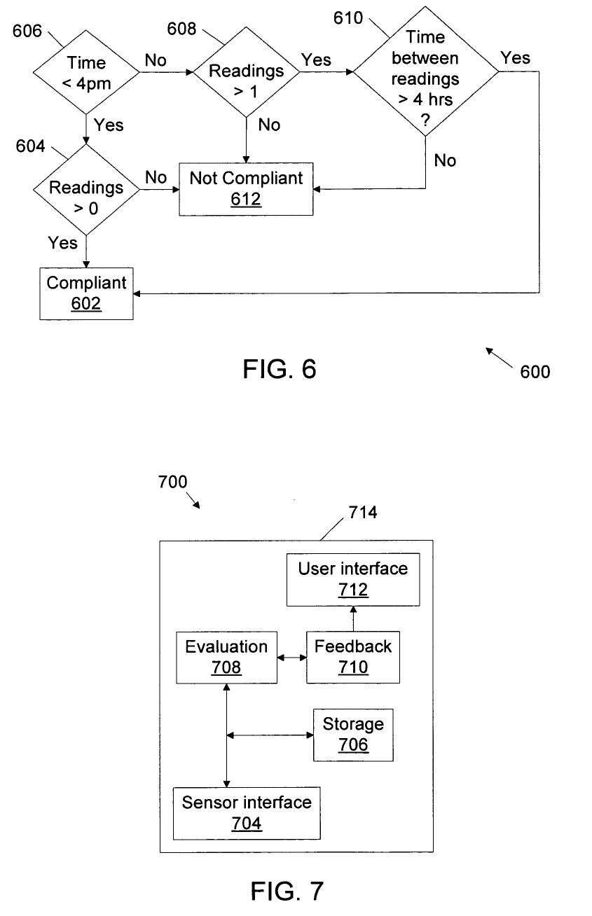

FIG. 6 shows an exemplary rule 600 that can be used to determine 512 if the patient is compliant with his self-monitoring regimen at a particular time of the day. An example rule 600 considers the player compliant 602 if at least one weight measurement, or reading, is received before 4:00 pm 604, 606, respectively. An example rule 600 also considers the patient compliant if, after 4:00 pm, two or more readings have been received 608, with a minimum of four hours between the first and the last 610. Otherwise, in the example embodiment, the patient or user is non-compliant 612. As described with relation to FIG. 4, rules such a rule 600 can be developed by medical experts for an individual patient, or a group of similar patients, and can be more detailed, and/or can depend on different variables, than shown for exemplary rule 600.

Referring again to FIG. 5, the application 500 can be activated 518 and deactivated 520 by a password, which can be entered when the application 500 is started and/or initiated. Other activation and deactivation schemes can be used, including a timing mechanism that activates the application 500 and blocks deactivation for a specified length of time, or by accessing another application, such as a web site, that can provide motivational support if the patient seeks to deactivate the application 500 prematurely. The password method can work best if the deactivation password is not known to the patient using the application(s) 500. In some embodiments, a password method can be useful in situations where an adult wishes to monitor the compliance of a child or adolescent.

The application 500 can motivate the patient to follow a prescribed self-monitoring regimen by rewarding compliance with access to other applications, such as games and/or lifestyle tools accessible through the PDA and/or processor executing the application 500. This approach can leverage the appeal and entertainment value of unrelated, generic computer applications, games and/or tools to motivate the patient without relying on special-purpose motivational applications.

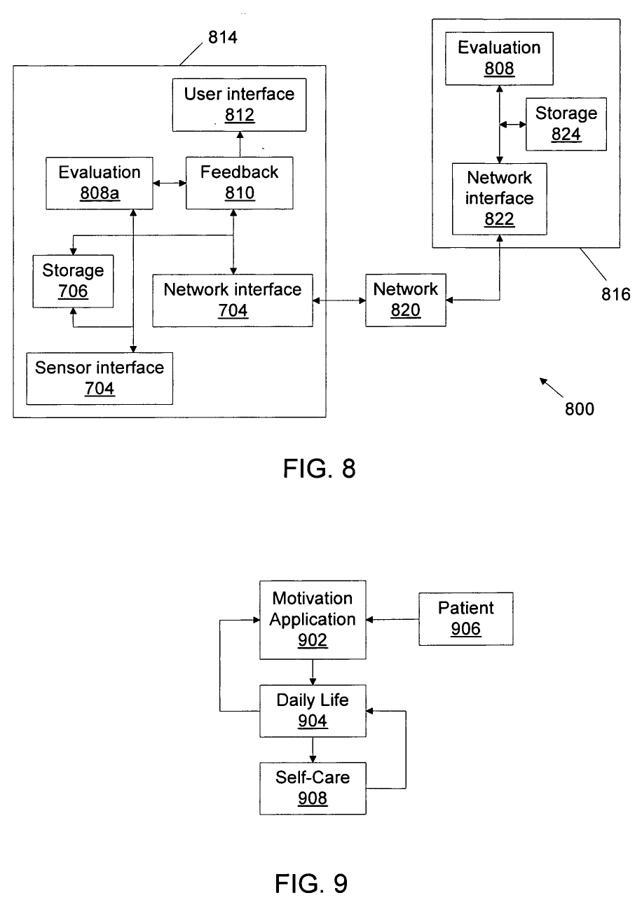

FIG. 7 illustrates a block diagram of a system 700 for implementing one or more of the methods described herein in a local evaluation approach. FIG. 8 illustrates a block diagram of a system 800 for implementing one or more of the methods described herein in a server evaluation approach. Systems 700, 800 receive usage and/or health relevant data directly from monitoring and/or therapeutic devices 702, 802, via respective sensor interfaces 704, 804, including ports such as infrared ports and/or wireless communication chips. Data can be stored in respective storage units 706, 806 and used by respective evaluation software 708, 808. Evaluation software 708, 808 can evaluate compliance and respective feedback software 710, 810 can reward compliance via respective interactive user interfaces 712, 812. For the illustrated FIG. 7 system 700, evaluation and feedback software 708, 710 can reside and execute on a local processor 714 to evaluate and reward compliance through the illustrated interactive user interface 712.

In a networked approach demonstrated by FIG. 8, a system 800 can include a processor 814, such as a wireless communication device, PDA, and/or cellular phone, and remote server 816. The processor 814 can include a network interface 818 for forwarding the data from a storage unit 806 to a server 816 via a network 820, such as a cellular network and/or the Internet. Data received at a server 816 from the network 820 via the interface 822 can be stored in a storage unit 824 of a server 816. Evaluation software 806 can use the data from the storage unit 824 to evaluate compliance and provide results, directions, content, and/or other information to a processor 814 over the network 820 via network interfaces 822 and 818. The results, directions, content, and/or other information can be stored or provided to feedback software 810. The illustrated feedback software 810 can reward compliance via an interactive user interface 812. For the illustrated system 800, evaluation software 806 can generally reside on the remote server 816, though portions can also reside on the processor 814.

It is clear that there are many ways to motivate and reward chronic disease patients, and/or others requiring or desiring monitoring, for compliance with prescribed self-care regimens. Motivation can be achieved by incorporating rewards into computer games and/or lifestyle tools, or by granting access to restricted features and/or applications. The disclosed methods and systems can be deployed on convenient processor platforms, including personal digital assistants, computerized watches, cellular phones and/or portable computers. The disclosed methods and systems provide real-time and automated evaluation of self-care compliance throughout the daily routine of the patient by receiving usage data directly from a compatible monitoring or therapeutic device.

The methods and systems disclosed herein can be used for motivating the correct and timely use of monitoring and therapeutic devices for a wide range of diseases, such as diabetes, asthma and other breathing disorders, hypertension and other cardiovascular conditions, obesity, hemophilia and other bleeding disorders, eating disorders, and depression and other mood disorders, and for a wide range of behavior modification and/or monitoring programs, such as self-medication programs, quitting smoking programs, weight loss programs, physical therapy, physical conditioning, etc. Compatible monitoring or therapeutic devices can include blood glucose level measuring devices, inhalation devices, electronic weighing scales, blood pressure measuring devices, heart rate monitoring devices, accelerometers, blood clotting measurement devices, voice recording devices, and medication dispensing devices. Therefore, the scope of the disclosed methods and systems are not limited by the examples given herein, but can include the full scope of the claims and their legal equivalents.

The methods and systems described herein are not limited to a particular hardware or software configuration, and may find applicability in many computing or processing environments. The methods and systems can be implemented in hardware or software, or a combination of hardware and software. The methods and systems can be implemented in one or more computer programs, where a computer program can be understood to include one or more processor executable instructions. The computer program(s) can execute on one or more programmable processors, and can be stored on one or more storage medium readable by the processor (including volatile and non-volatile memory and/or storage elements), one or more input devices, and/or one or more output devices. The processor thus can access one or more input devices to obtain input data, and can access one or more output devices to communicate output data. The input and/or output devices can include one or more of the following: Random Access Memory (RAM), Redundant Array of Independent Disks (RAID), floppy drive, CD, DVD, magnetic disk, internal hard drive, external hard drive, memory stick, or other storage device capable of being accessed by a processor as provided herein, where such aforementioned examples are not exhaustive, and are for illustration and not limitation.

The computer program(s) can be implemented using one or more high level procedural or object-oriented programming languages to communicate with a computer system; however, the program(s) can be implemented in assembly or machine language, if desired. The language can be compiled or interpreted.

As provided herein, the processor(s) can thus be embedded in one or more devices that can be operated independently or together in a networked environment, where the network can include, for example, a Local Area Network (LAN), wide area network (WAN), and/or can include an intranet and/or the internet and/or another network. The network(s) can be wired or wireless or a combination thereof and can use one or more communications protocols to facilitate communications between the different processors. The processors can be configured for distributed processing and can utilize, in some embodiments, a client-server model as needed. Accordingly, the methods and systems can utilize multiple processors and/or processor devices, and the processor instructions can be divided amongst such single or multiple processor/devices.

The device(s) or computer systems that integrate with the processor(s) can include, for example, a personal computer(s), workstation (e.g., Sun, HP), personal digital assistant (PDA), handheld device such as cellular telephone, laptop, handheld, or another device capable of being integrated with a processor(s) that can operate as provided herein. Accordingly, the devices provided herein are not exhaustive and are provided for illustration and not limitation.

References to “a microprocessor” and “a processor”, or “the microprocessor” and “the processor,” can be understood to include one or more microprocessors that can communicate in a stand-alone and/or a distributed environment(s), and can thus can be configured to communicate via wired or wireless communications with other processors, where such one or more processor can be configured to operate on one or more processor-controlled devices that can be similar or different devices. Use of such “microprocessor” or “processor” terminology can thus also be understood to include a central processing unit, an arithmetic logic unit, an application-specific integrated circuit (IC), and/or a task engine, with such examples provided for illustration and not limitation.

Furthermore, references to memory, unless otherwise specified, can include one or more processor-readable and accessible memory elements and/or components that can be internal to the processor-controlled device, external to the processor-controlled device, and/or can be accessed via a wired or wireless network using a variety of communications protocols, and unless otherwise specified, can be arranged to include a combination of external and internal memory devices, where such memory can be contiguous and/or partitioned based on the application. Accordingly, references to a database can be understood to include one or more memory associations, where such references can include commercially available database products (e.g., SQL, Informix, Oracle) and also proprietary databases, and may also include other structures for associating memory such as links, queues, graphs, trees, with such structures provided for illustration and not limitation.

References to a network, unless provided otherwise, can include one or more intranets and/or the internet. References herein to microprocessor instructions or microprocessor-executable instructions, in accordance with the above, can be understood to include programmable hardware.

References and/or the use of the articles “a” or “an”, unless otherwise specified herein, can be understood to include references to one or more of the noun to which the articles refer. Accordingly, throughout the entirety of the present disclosure, use of the articles “a” or “an”, unless otherwise provided, is for convenience only and is not intended to limit the noun in the singular. Use of the article “the” is also for convenience, and is not intended to limit the modified noun in the singular, and/or otherwise indicate that the disclosed methods and systems are limited to the description/depiction of the modified noun.

Although the methods and systems have been described relative to a specific embodiment thereof, they are not so limited. Obviously many modifications and variations may become apparent in light of the above teachings.

Many additional changes in the details, materials, and arrangement of parts, herein described and illustrated, can be made by those skilled in the art. Accordingly, it will be understood that the methods and systems provided herein are not to be limited to the embodiments disclosed herein, can include practices otherwise than specifically described, and are to be interpreted as broadly as allowed under the law.

1. A method, comprising:
receiving data associated with the compliance of a user with a self-care regimen,
evaluating the data to determine a level of compliance of the user with the self-care regimen, and
using at least one executable processor application to provide the user with an incentive based on the determined level of compliance.

2. The method of claim 1, where the incentive includes changing a condition of a virtual entity, where the changed condition is associated with the determined level of compliance.

3. The method of claim 2, where the changed condition is associated with the user's condition based on the determined level of compliance and the self-care regimen.

4. The method of claim 1, where the incentive includes a reward to the user.

5. The method of claim 5, where the reward includes access to at least one of: at least one additional processor application, at least one enhanced features of a processor application, and a change in a user score.

6. The method of claim 1, where the incentive includes a change in a user score, where the user score affects at least one of an activity, a physical condition, and a characteristic of a virtual entity associated with the at least one processor application.

7. The method of claim 6, where the score affects at least one of a health and a happiness of a virtual pet.

8. The method of claim 1, where receiving data includes receiving data from at least one of: a blood glucose level measuring device, an inhalation device, an electronic weighing scale, a blood pressure measuring device, a heart rate monitoring device, an accelerometer, a blood clotting measurement device, a voice recording device, and a medication dispensing device.

9. The method of claim 1, where evaluating includes determining compliance with at least one of: a diabetes management self-care regimen, a breathing disorder self-care regimen, a weight management self-care regimen, a cardiovascular condition self-care regimen, a hypertension self-care regimen, a physical activity self-care regimen, a bleeding disorder self-care regimen, a mood disorder self-care regimen, and a self-medication self-care regimen.

10. The method of claim 1, wherein evaluating comprises:
establishing at least one rule describing respective conditions to be satisfied in attaining respective levels of compliance with the self-care regimen, and
evaluating at least one of the rules based on the data.

11. The method of claim 1, where the self-care regimen is a medical regimen.

12. The method of claim 1, where the at least one executable processor application resides on at least one of: a personal digital assistant, a cellular telephone, a wireless personal communications device, a portable video game device, and a digital watch.

13. A computer game tangibly stored on a computer-readable medium and operable to cause a processor to:
receive data relevant to the compliance of a user with a self-care regimen,
evaluate the data to determine a level of compliance with the self-care regimen,
update at least one of an activity, a physical condition, and characteristic of at least one interactive virtual entity, based on the determined level of compliance, to obtain a condition of the virtual entity, and,
display the virtual entity to the user to convey the condition of the virtual entity to the user.

14. The computer game of claim 13, further comprising instructions to provide a choice of actions to the user, the actions for modifying the condition of the virtual entity.

15. The computer game of claim 13, wherein the instructions further comprise instructions to mark the virtual entity for deletion based on a comparison of the condition to a threshold.

16. The computer program of claim 14, wherein the instructions to update comprise instructions to:
determine a time interval since the condition was obtained, and
update the condition based on the time interval.

17. The computer game of claim 13, wherein the instructions to display further comprise instructions to convey the condition of the virtual entity consistent with the self-care regimen effects on the user.
